(19) United States
(12) Patent Application Publication
(10) Pub. No.: US 2014/0212468 A1
(43) Pub. Date: Jul. 31, 2014

(54) PREPARATION METHOD OF AN IMPLANT COMPRISING DRUG DELIVERY LAYER AND IMPLANT COMPOSITION FOR LIVING DONOR TRANSPLANTATION COMPRISING THE SAME

(71) Applicant: Industry-Academic Cooperation Foundation, Dankook University, Yongin-si (KR)

(72) Inventors: Hae-Won Kim, Cheonan-si (KR); Kapil Dev Patel, Cheonan-si (KR)

(73) Assignee: Industry-Academic Cooperation Foundation, Dankook University, Yongin-si (KR)

(21) Appl. No.: 14/045,109

(22) Filed: Oct. 3, 2013

(30) Foreign Application Priority Data
Jan. 29, 2013 (KR) 10-2013-0010087

(51) Int. Cl. A61L 31/12; A61L 31/16

(52) U.S. Cl. CPC A61L 31/128; A61L 31/16
USPC 424/423; 204/499

(57) ABSTRACT

The present invention relates to a preparation method of implant comprising drug delivery layer and implant composition for living donor transplantation comprising the same, and more specifically, a preparation method of implant comprising drug delivery layer comprising preparing chitosan-bioactive glass composite solution; preparing drug-containing complex coating composition by adding drug in the chitosan-bioactive glass composite solution; and preparing drug delivery layer by electrophoresis of the complex coating composition on the surface of implant, and implant composition for living donor transplantation comprising the same. The implant composition according to the present invention is able to deliver the drug, and therefore to prevent inflammation, which may occur after the surgery, as well as to promote recovery depending on the type of drugs contained. Thus, a preparation method of implant comprising drug delivery layer and implant composition for living donor transplantation comprising the same according to the present invention can be usefully applied to the bone transplantation field and bone transplant material.

### BACKGROUND OF THE INVENTION

1. Field of the Invention

The present invention relates to a preparation method of an implant comprising a drug delivery layer and implant composition for living donor transplantation comprising the same, and more specifically, to a preparation method of an implant comprising a drug delivery layer comprising preparing a chitosan-bioactive glass composite solution; preparing a drug-containing complex coating composition by adding drugs to the chitosan-bioactive glass composite solution; and preparing a drug delivery layer by electrophoretic deposition of the complex coating composition on the surface of the implant, and an implant composition for living donor transplantation comprising the same.

2. Description of the Prior Art

Bone transplantation is the second most commonly conducted operation next to blood transfusion, and is being performed in a number of areas to treat bone defects commonly seen clinically in the orthopedic field. According to the report, more than 500,000 bone transplantations per year are performed in the United States, and approximately 2.2 million per year are performed in the world. Bone transplant material is used for stimulating bone growth in the region where bone is missing due to pathological or physiological causes.

Action of bone transplant material can be classified into osteoconduction, osteoinduction, osteogenesis depending on the mechanism. Osteoconduction is the formation of a bone by migrations of osteoblast from the surrounding osseous tissue to the site of the transplanted bone and the deposition of minerals, which requires surrounding osseous tissue or differentiated mesenchymal cells. Osteoconduction material, if transplanted to the other areas such as subcutaneous tissue, cannot initiate bone growth. When transplanted to osseous tissue or soft tissue, osteoconduction material is absorbed and replaced by osseous tissue through the similar process as creeping substitution. Osteoinduction is the process of formation of new osseous tissue by effects of bone transplant material on the differentiation of undifferentiated mesenchymal cells into osteoprecursor cell, in which bone morphogenetic protein (BMP) is generally known to be involved. When transplanted to other tissues such as subcutaneous tissue, bone formation is possible. Osteoinductive transplant material has a significant effect on the process of bone remodeling as well. Meanwhile, osteogenesis refers to the formation of osseous tissue by living cells in the transplant material, and autologous bone is the only material for osteogenesis.

The materials used for bone transplantation are autologous bone, allogenic bone, xenogenic bone, etc. and in recent years, a variety of artificial bone substitutes have been developed and used. Ideally, bone transplant materials need to meet requirements such as induction of bone formation, biocompatibility with host, ease of the collection and handling, reduction of costs, but each of currently used bone transplant materials has some degree of drawback.

Autologous bone is the most viable material, having all three characteristics of osteoconduction, osteoinduction, osteogenesis for synostosis and has a higher stability and transplantation rate than other bone transplant materials. However, it can cause another defects and complications such as infection in the donor from whom transplant bone is collected, requires additional surgery time, and often has the disadvantage of difficulty in getting a sufficient amount of bone. In order to overcome these disadvantages, research for substitute bone transplant materials have been widely carried out.

Currently, allogenic bone, which is the most commonly used alternative to autologous bone, is tissue obtained from the same species as the host, and available in many forms such as particle, gel, putty, etc. by freezing, freeze-drying, and demineralized freeze-drying, etc. Allogenic bone can be obtained in a relatively large amount and has the advantage of adjustable shape or bone density, etc. by using certain parts of the skeleton or by processing. Allogenic bone as a bone transplant material has superior osteoconductivity, but no ability for osteogenesis since bone cells don't survive, and extremely limited osteoinductivity. In addition, it is limited in supply, has restrictions as an ethical issue, and has the disadvantage of risks such as spread of contaminants, toxins or infections. Also, even with a thorough investigation of donors and various examinations, allogenic bone has the possibility of contagion of infectious diseases caused by viruses, and, if processed in various ways in order to reduce the risks, may have modified biological and mechanical properties, which can weaken mechanical strength, and adversely affect osteoconductivity and osteoinductivity.

Meanwhile, xenogenic bone transplantation is used restrictively to process the bones of animals such as cows and pigs for the purpose of transplanting them into the human body, but the use thereof is being gradually reduced due to problems such as immune response and spread of infectious diseases.

Thus, due to the problems related to autologous bones, allogenic bones and xenogenic bones, in recent years interest in artificial bone substitutes which can provide biocompatibility and safety for bone transplantation has increased and many studies are being actively carried out.

Artificial bone substitutes are required to meet the following requirements.
1) Does not cause infection and no or minimal antigen.
2) Does not break during surgery, easy to handle and excellent in vivo absorbency.
3) Has sufficient mechanical strength to withstand the pressure of surrounding tissues after surgery, and maintains constant volume until osteo tissue is fully formed and mature in substrate.
4) Has appropriate surface roughness in order to attach well to existing tissue, and porosity to allow the proper spread of nutrients or excrement for adhesion, growth and differentiation of cells.
5) Possesses biocompatibility for additional products by decomposition of artificial bone substitutes.

Currently, as artificial bone materials, metallic materials such as titanium, stainless steel alloys, cobalt-chrome alloys, bio-inert ceramic materials such as alumina, zirconia, or bioactive ceramic materials such as hydroxyapatite are widely used. Hydroxyapatite, which is the most commonly used, has the same active ingredient and structure as bones or teeth in the human body, is used in the form of powder, dense body, or coating on metal in the medical field, and has excellent biocompatibility and bioaffinity causing the osteoconduction of the surrounding bone, and therefore research for developing the same as an artificial bone material and applying it experimentally and clinically has been actively conducted. However, despite the excellent biocompatibility, due to susceptibility to damage and distinctive brittleness of ceramic which is easily breakable, it has limited application as a bone substitute. In addition, other commonly used high-strength ceramic materials and metallic materials are characterized by high mechanical strength, but have the drawback of inducing bone regeneration due to low biocompatibility. Therefore, in order to resolve the above problems, a method of coating the material with excellent biocompatibility on the surface of metal or bio-inert ceramic is generally performed.

As a method for coating biocompatible materials, plasma spraying was used most often, which a method of melting the biocompatible material in a high-temperature plasma region of 20,000° C. to 30,000° C. and welding the same to the surface of a metal or bio-inert ceramic. The coating layer coated by plasma spraying has higher adhesion strength compared to the coating layer coated by chemical vapor deposition, sputtering, etc. but still has the problem of easy destruction of the coating layer between the surface of the metal and the surface of the coating layer. In recent years, methods of precipitating the biocompatible materials on the surface of metal by depositing metallic transplant body in a solution consisting of calcium and phosphate ions or producing a biocompatible material layer by modifying the surface and deposit in simulated body fluid (SBF) are being developed, but the method of coating biocompatible materials using simulated body fluid has the disadvantage of a long deposition time.

On the other hand, electrophoretic deposition (EPD) is one of the more useful and effective coating methods, which is mainly used due to reasons of simplicity and cheapness. Electrophoretic deposition has the advantage of preparing a highly homogeneous coating layer with a variable thickness, ranging from 100 μm to 0.3 μm. In addition, in electrophoretic deposition, either anodic or cathodic treatment can be applied according to the charge of particles or molecules to be deposited, which provides the advantage of easy control of coating composition and commercial use.

Meanwhile, ways for coating the surface with medications such as antibiotics or protein such as growth factor or as insulin are being studied in order to promote recovery by reducing the recovery time of damaged areas, the time required for stable combination of implant or artificial hip joints, and the period of osteo integration through prevention of acute inflammation which may occur at the beginning of the post-surgery period after placing the implant or artificial hip joint into the human body.

Currently, the methods for coating the surface of the implant with a drug can be divided into three main technologies.

First is a method of coating the surface of implant with a functional polymer mixed with a drug. The functional polymer coating has the problems of easy deterioration, decomposition and poor biocompatibility.

Second is a method of forming a coating layer biocompatible material on the surface of implant and adsorbing the drug physically on top thereof. The physical adsorption process has a problem of difficulty in controlling the release rate of the drug adsorbed on the surface.

Third is the method of complexing hydroxyapatite and drugs concurrently using biomimetic coating, by means of coating the surface of implant with hydroxyapatite crystals generated by precipitating the Ca²⁺, PO₄²⁻ ions from an aqueous solution comprising the Ca and P composition having appropriate pH, wherein the addition of drug to the said aqueous solution enables the simultaneous coating of hydroxyapatite and drug. Since the biomimetic coating method utilizes the precipitation of ions, the deposition rate of the coating layer is very slow, achieving less than 0.5 μm per hour, the coating process is complex, and the concentration of the drug in the coating layer is difficult to control accurately, and in addition, it is difficult to add a drug with high concentration and the bonding between the coating layer and the surface of metallic material is low, and therefore there is a problem of significantly limited industrial application.

Hereupon, while studying the composition for living donor transplantation with high binding affinity with the substrate, excellent biocompatibility and osteogenesis effect, the present inventors identified that as the result of forming the coating layer on the metallic substrate with drug-containing chitosan-bioactive glass complex coating composition through electrophoretic deposition, the coating layer forms a homogeneous structure as well as exhibits the excellent effect of cell proliferation, apatite formation, and drug delivery, to thereby complete the present invention.

### SUMMARY OF THE INVENTION

The objective of the present invention is to provide the preparation method for an implant with excellent biocompatibility and osteogenesis capability, comprising a drug delivery layer with capability of delivering the drug.

Another objective of the present invention is to provide the implant composition for living donor transplantation, comprising the implant comprising a drug delivery layer prepared by the preparation method.

### DETAILED DESCRIPTION OF THE INVENTION

In order to achieve the objectives, the present invention provides a preparation method of an implant comprising a drug delivery layer comprising 1) preparing a chitosan-bioactive glass composite solution by dispersing chitosan solution in solvent and adding surface aminated bioactive glass nanoparticles (step 1); 2) preparing a drug-containing complex coating composition by adding the drug to the chitosan-bioactive glass composite solution (step 2); and 3) preparing a drug delivery layer by electrophoretic deposition of the complex coating composition on the surface of the implant (step 3).

As used herein, the term “chitosan” refers to the natural polymer which can be obtained from the exoskeleton of insects, crustaceans, and fungi. In general, chitosan is widely distributed in nature and can be obtained by deacetylation of chitin which is polysaccharide.

As used herein, the term “bioactive glass” refers to a material with excellent bioactivity, which is the capability of chemical bonding with in vivo tissues without any resulting toxicity, inflammation of negative immune responses, by forming the layer of hydroxyapatite, which has a similar composition to the bone, on the surface after transplantation into the body.

Step 1 is to prepare a chitosan-bioactive glass composite solution by dispersing chitosan solution in solvent and adding the surface aminated bioactive glass nanoparticles in order to prepare a chitosan-surface aminated bioactive glass nanoparticle composite solution by mixing chitosan solution and surface aminated bioactive glass nanoparticles. Preferably, the chitosan solution is chitosan dissolved in acetic acid or hydrochloric acid solution. In addition, the solvent may be the co-solvent which is the mixture of ethanol and water in a volume ratio of 1:1 to 1:9, and preferably, the co-solvent may have a mixture with a volume ratio of 1:4.

It is preferable that the surface aminated bioactive glass nanoparticles are prepared through the following process.
1) Preparing calcium nitrate—template mixture solution by preparing PEG template solution and adding calcium nitrate;
2) Preparing a reaction product by adding TEOS solution in calcium oxide-template mixture solution, sonicating and stirring;
3) Preparing the bioactive glass nanoparticles by centrifuging the reaction product, washing and calcining; and
4) Adding APTES followed by refluxing after adding the prepared bioactive glass particles in a solvent followed by dispersing.

It is preferable that the surface aminated bioactive glass nanoparticles comprise silica (SiO₂) and calcium oxide (CaO) in a molar ratio of 85:15.

Step 1) is to prepare calcium nitrate-template mixture solution by preparing PEG template solution and adding calcium nitrate in the solution in order to prepare the bioactive glass nanoparticles. The polymer template solution can be prepared by dissolving the polymer template in ethanol. Preferably, the polymer template is PEG. In addition, pH of the polymer template solution can be adjusted by adding ammonium hydroxide.

Step 2) is to prepare a reaction product by sonicating and stirring while adding TEOS solution in calcium nitrate-template mixture solution in order to form the target mineral. The TEOS solution is preferably TEOS dissolved in ethanol, but is not limited thereto. In addition, the sonication can be performed with intensity of 10 kHz to 40 kHz and power of 100 W to 1000 W for 10 minutes to 20 minutes at cycle of 10 sec on/10 sec off, but is not limited thereto.

Step 3) is to centrifuge the reaction product at 8,000 rpm to 15.000 rpm, wash with distilled water and ethanol, and then filter and calcine in order to obtain the bioactive glass nanoparticles by removing PEG from the reaction product. The calcination is preferably performed at the temperature of 500° C. to 800° C. for 1 to 10 hours, but not limited thereto.

Step 4) is to add APTES, which is the amine compound, and refluxing after dispersing the bioactive glass nanoparticles in a solvent in order to obtain the surface aminated bioactive glass nanoparticles by amine-functionalizing the surface of bioactive glass nanoparticles prepared in the step 3). Refluxing can be performed at the temperature of 80° C. to 90° C. for 12 hours to 24 hours, but is not limited thereto.

Also, the step for washing and drying may be additionally included after the refluxing. Drying can be performed preferably at the temperature of 60° C. or 90° C. for 12 hours to 72 hours, but is not limited thereto.

Step 2 is to prepare drug-containing complex coating composition by adding drug in the chitosan-bioactive glass composite solution prepared in the step 1. The complex coating composition is preferably in the range of pH 3.6 to pH 3.1, and the pH is preferably adjusted with the acetic acid or sodium hydroxide. In addition, the drug is preferably antibiotic, anti-inflammatory drug, anticancer drug, or bone differentiation drug, but is not limited thereto.

Step 3 is to prepare a drug delivery layer by electrophoretic deposition of the drug-containing complex coating composition in order to form the drug delivery layer on the surface of implant. The electrophoretic deposition is performed preferably with the DC voltage of 20 V to 80 V for 5 minutes to 10 minutes. The thickness of the drug delivery layer is preferably 2 μm to 50 μm. If the thickness of drug delivery layer is less than 2 μm, the drug delivery efficacy of coating layer can be incomplete. In addition, the thickness over 50 μm can cause the problem of the detachment of coating film.

In addition, the present invention provides the implant composition for living donor transplantation, comprising the implant prepared by the above preparation method.

### Effect of the Invention

The preparation method of an implant using electrophoretic deposition according to the present invention has the effects of homogeneous coating of the substrate using the composition in the form of liquid (granule) and easy adjustment of the thickness of the coating layer.

In addition, the implant composition for living donor transplantation comprising the drug delivery layer according to the present invention is able to deliver the drug, and therefore to prevent inflammation, which may occur after surgery, as well as to promote recovery depending on the type of drugs contained.

Thus, a preparation method of an implant comprising drug delivery layer and implant composition for living donor transplantation comprising the same according to the present invention can be usefully applied to the bone transplantation field and bone transplant material.

### BRIEF DESCRIPTION OF THE DRAWINGS

FIG. 1 is a graph showing the weight change according to (a) power, (b) deposition time and (c) content change of the surface aminated bioactive glass particles during the electrophoretic deposition process according to an example of the present invention.

FIG. 2 is a graph showing the zeta potential of the bioactive glass nanoparticles and the surface aminated bioactive glass nanoparticles according to an example of the present invention.

FIG. 3 shows the results of the XRD analysis of (a) the surface aminated bioactive glass nanoparticles and (b) chitosan-bioactive glass compound coating layer (including control) according to an example of the present invention.

FIG. 4 shows the results of the FT-IR analysis of (a) the bioactive glass nanoparticles and the surface aminated bioactive glass nanoparticles and (b) chitosan-bioactive glass compound coating layer (including control) according to an example of the present invention.

FIG. 5 shows the TEM images of (a) the surface aminated bioactive glass nanoparticles and (b) chitosan-bioactive glass complex coating composition (granular solution) according to an example of the present invention.

FIG. 6 shows the results of the turbidity analysis of the chitosan-bioactive glass complex coating composition (granular solution) according to an example of the present invention.

FIG. 7 shows the TGA results of chitosan (control) and chitosan-bioactive glass compound coating layer according to an example of the present invention.

FIG. 8 shows the SEM images of (a) chitosan (CH), (b) chitosan-5 wt % bioactive glass compound coating layer (CH-5BGn), (c) chitosan-10 wt % bioactive glass compound coating layer (CH-10BGn), (d) chitosan-15 wt % bioactive glass compound coating layer (CH-15BGn) and (e) the coating layer scratched off from chitosan-5 wt % bioactive glass compound coating layer according to an example of the present invention

FIG. 9 shows the time-dependent degradation rates of chitosan (CH) and chitosan-bioactive glass compound coating layer (CH-BGn) according to an example of the present invention.

FIG. 10 shows the analysis results of time-dependent capability of apatite formation of chitosan (CH) and chitosan-bioactive glass compound coating (CH-BGn) according to an example of the present invention.

FIG. 11 shows the results of (a) SEM, (b) XRD and (c) FT-IR analysis of property change after culturing in simulated body fluid (acceleration medium) according to an example of the present invention.

FIG. 12 shows the results of analysis of (a) SEM (culture day 3) and (b) cell proliferation (growth) after culturing MC3T3-E1 cells in chitosan (CH) or chitosan-10 wt % bioactive glass compound coating layer (CH-10BGn) according to an example of the present invention.

FIG. 13 shows the results of the gene expression of (a) Cod I, (b) ALP, (c) BSP, (d) OPN and (e) OCN after culturing the cells in each coating layer (Ti (titanium), CH (chitosan) and CH-10BGn (chitosan-10 wt % bioactive glass compound coating layer)) according to an example of the present invention.

FIG. 14 shows the results of analysis of (a) Na-ampicillin (model drug) release and (b) antibacterial activity according to an example of the present invention.

### DETAILED DESCRIPTION OF THE PREFERRED EMBODIMENTS

Hereinafter, the present invention is described in more detail through providing Examples and Experimental Examples. However, these Examples and Experimental Examples are merely meant to illustrate, but in no way to limit, the claimed invention.

### Example

### Preparation of Drug-Containing Implant

Chitosan of middle molecular weight (MW=200,000 Da, 85% deacetylation), acetic acid (≧99%), polyethylene glycol (PEG, Mn: 10,000), calcium nitrate (Ca(NO₃)₂.4H₂O), ammonium hydroxide (28% NH₃ in water, ≧99.99% metal basis), TEOS(C₈H₂₀O₄Si, 98%), anhydrous methanol (CH₄O, 99.8%), anhydrous toluene (C₇H₈, 99.8%) and APTES (C₉H₂₃NO₃Si, ≧98%) were purchased from Sigma-Aldrich (USA), and used without purification. For coating, pure titanium (Ti) (cp Ti, Senulbio Biotech, Korea) in the form of square plate (10 mm×10 mm×1 mm) was used as the metal substrate.

1) Preparation of the Surface Aminated Bioactive Nanoparticles

Through the preliminary experiments, Si and Ca with the ratio of 85 mol %:15 mol % were identified to show excellent biological activity (ex vivo experiments), while maintaining excellent spherical nanoparticle morphology. Therefore, the binary bioactive glass nanoparticles of 85SiO₂-15CaO were prepared by adjusting the Si and Ca ratio to 85 mol %:15 mol %.

First, PEG of 5 g was dissolved in ethanol of 150 mL while stirring at 40° C., and then the clear mixture was obtained by adding ammonium hydroxide of 30 mL and calcium nitrate (Ca(NO₃)₂.4H₂O) of 358 g. Separately from this, the solution was prepared by dissolving TEOS 2 mL in ethanol 20 mL, and then the resultant was added to the mixture of PEG and calcium nitrate in drops, and was homogenized by sonicating with the ultrasonic generator (LH700S ultra-sonic generator; Ulsso Hitech, Korea). At this time, sonication is performed at the intensity of 20 kHz, under the power condition of 700 W (35% of the output for 10 minutes, on/off cycle of 10s/10s) and then with 220 W for 20 minutes at the on/off cycle of 10s/10s. Subsequently, a white gel precipitate was obtained by string vigorously the mixture for 24 hours at room temperature, centrifuged at 10,000 rpm centrifugation, washed with distilled water and ethanol, and then was filtered. The bioactive glass nanoparticles were prepared by treating the white powder obtained as a result with heat at 600° C. for 5 hours.

In order to aminate the surface, the prepared bioactive glass nanoparticles were reacted with APTES. First, homogeneous solution was obtained by adding the bioactive glass nanoparticles of 0.1 g in toluene of 50 mL and sonicating for 30 minutes. APTES of 1 mL was added to the solution, refluxed at 80° C. for 24 hours, centrifuged at 10,000 rpm for 5 minutes, and washed with toluene and ethanol. The surface aminated bioactive glass nanoparticles were obtained by drying the product in an oven of 80° C. for 24 hours.

2) Preparation of Chitosan-Surface Aminated Bioactive Glass Complex Coating Composition (CH-BGn) and Coating Layer Using the Same

First, chitosan was dissolved in acetic acid solution of 1% and dispersed in ethanol/water co-solvent (25% v/v water) to 1 g/l. Then, chitosan-bioactive glass complex coating composition was prepared by dispersing the surface aminated bioactive glass nanoparticles, which prepared in example 1), at the various concentrations of 0 wt % (control), 5 wt %, 10 wt %, 15 wt % and 20 wt % through sonication for 30 minutes. The homogeneous distribution of the surface aminated bioactive glass nanoparticles in the chitosan solution was identified by turbidity test (Smart Scientific analysis, Turbiscan, Korea). Optical transmittance (%) of the complex coating composition was observed at every hour for 24 hours, and the morphology was identified with TEM.

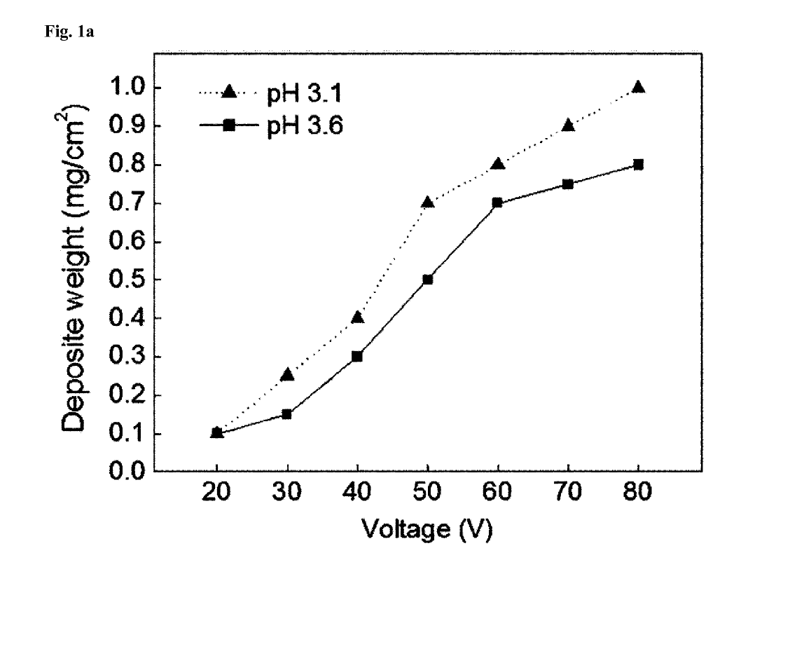

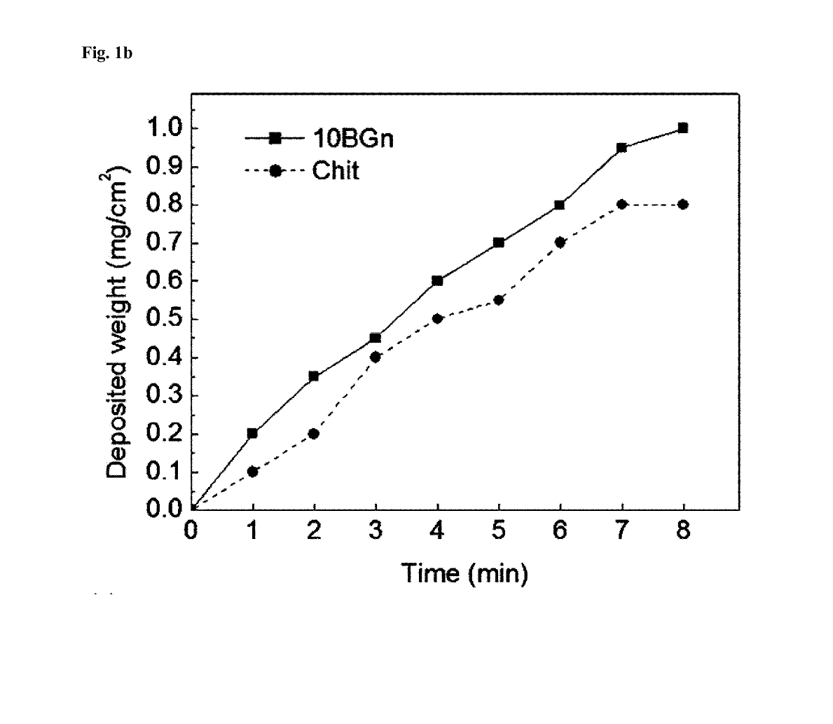

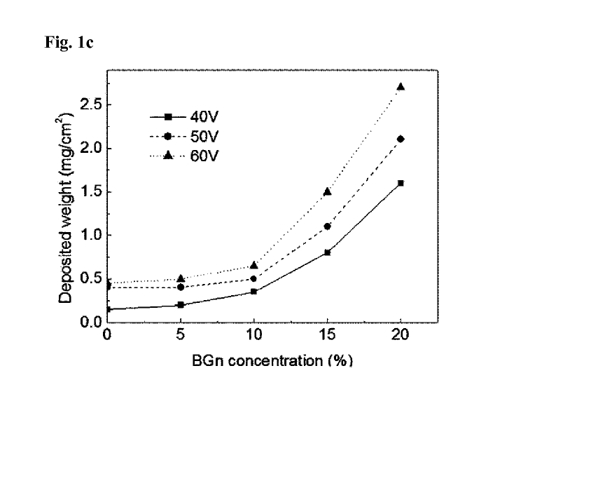

The coating layer was formed by electrophoretic deposition of each prepared complex coating composition on the surface of the metal substrate. In this case, since the complex coating composition is positively charged, the metal substrate was used as the cathode. Titanium (Ti) substrate was placed on the cathode, and the cathode-anode distance was maintained at 11 mm. Ultrasonic bath was degased, and DC voltage was applied using power supply (N5771A, 300V/5A; Agilent Technologies). Electrophoretic deposition process was performed as changing the pH, coating voltage and coating time in various conditions, in order to identify the optimal conditions for forming the coating layer according to the pH of coating composition, coating voltage and coating. And, while performing electrophoretic deposition process, the weight gain of the coating layer according to the change in each parameter was observed. Since when electrophoretic deposition was performed at the pH higher than 3.6, non-homogeneous coating morphology was observed, the pH of the coating composition was adjusted in the range of 3.1 to 3.6 using acetic acid and sodium hydroxide solution. The DC voltage was varied in the range of 20 V-80V, and the deposition time was set up to 8 minutes. Electrophoretic deposition was performed at atmospheric condition, and the each coating sample (metal substrate on which coating layer was formed) was taken, washed gently and dried for the test, after the deposition process was completed. The coating layer consisting of the surface aminated bioactive glass nanoparticles of 0 wt % (control), 5 wt %, 10 wt %, 15 wt % or 20 wt % was written as CH (control), CH-5BGn, CH-10BGn, CH-15BGn, and CH-20BGn, respectively, and the observation results of weight gain were shown in FIG. 1.

As shown in FIG. 1a, the weight of CH-10BGn increased with the voltage (increase from 20V to 80V). The weight gain showed a more noticeable increase at pH 3.1, which is more acidic than pH 3.6. This shows that decrease in pH (acidity) increases the property of positive potential of the chitosan molecules and bioactive glass nanoparticles.

As shown in FIG. 1b, the weight of the coating layer was found to increase almost linearly during the coating time.

In addition, as shown in FIG. 1c, the increase in weight of the coating layer was found to be not linear but rather exponential, as the increase in the content of the surface aminated bioactive glass nanoparticles. As a result, the addition of the surface aminated bioactive glass nanoparticles was identified to increase the weight of the composition (at constant volume).

3) Preparation of Drug-Containing Complex Coating Composition and Drug Delivery Layer.

Na-ampicillin was used as a model drug for loading and release test of the drug for the electrophoretic deposition coating. Pure chitosan or chitosan-10 wt % surface aminated bioactive glass nanoparticles compound composition was prepared with 1% acetic acid/distilled. Na-ampicillin of two different amounts (5 mg; low Amp and 10 mg; high Amp) was dissolved with the fixed amount of chitosan at 100 mg, and electrophoretic deposition was performed at 40 kV for 5 minutes. Titanium (Ti) substrate was placed on the cathode and cathode-anode distance was maintained at 11 mm. Ultrasonic bath was degased, and DC voltage was applied using a power supply (N5771A, 300V/5A; Agilent Technologies). Electrophoretic deposition was performed with the voltage of 40 V for 5 minutes at atmospheric condition, CH (pure chitosan, high Amp) and CH-10BGn (chitosan-10 wt % surface aminated bioactive glass nanoparticles, low Amp or high Amp), which are the samples of each drug delivery layer (metal substrate on which drug-containing coating layer formed), were taken, washed gently and dried for the subsequent test, after the deposition process was completed.

### Experimental Example 1

Analysis of Physico-Chemical Characteristics

Analysis of the physical and chemical characteristics of the surface aminated bioactive glass nanoparticles of Example 1) and the chitosan-bioactive glass compound coating layer of Example 2) (CH-5BGn, CH-10BGn, CH-15BGn and CH-20BGn) was performed.

1) Z-Potential Analysis

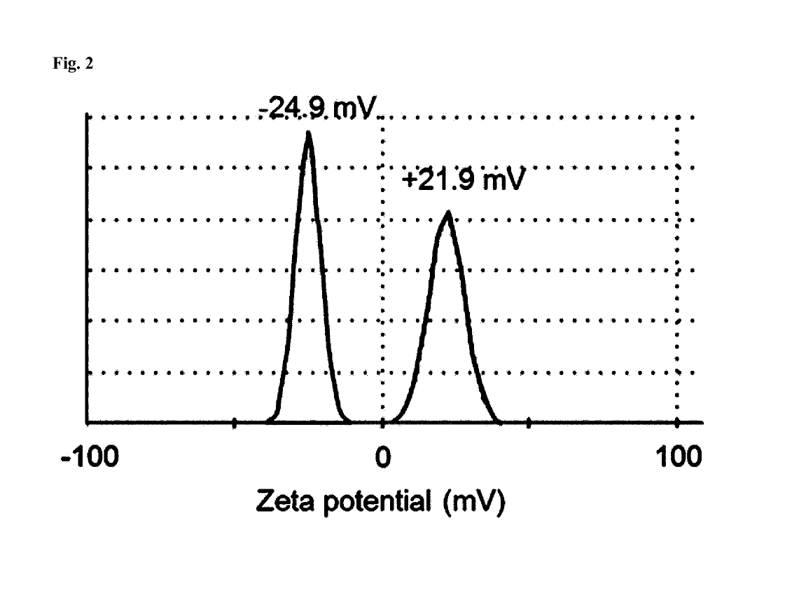

In order to identify changes in the surface potential of the bioactive glass nanoparticles before and after surface-amination of Example 1), zeta potential analysis was performed. For zeta potential analysis, the electrophoretic mobility was measured using zeta potential meter (Zetasizer Nano, Malvern, UK) under the condition of pH 7.4 and temperature 25° C. The measured electrophoretic mobility was converted to zeta potential using the Smoluchowski equation. The measurement results are shown in FIG. 2.

As shown in FIG. 2, the bioactive glass nanoparticles was changed from the negative potential (−24.9 mV) to the positive potential (+21.9 mV) after surface amination. This represents the successful amination of the surface of the bioactive glass nanoparticles.

2) XRD (X-Ray Diffraction) Analysis

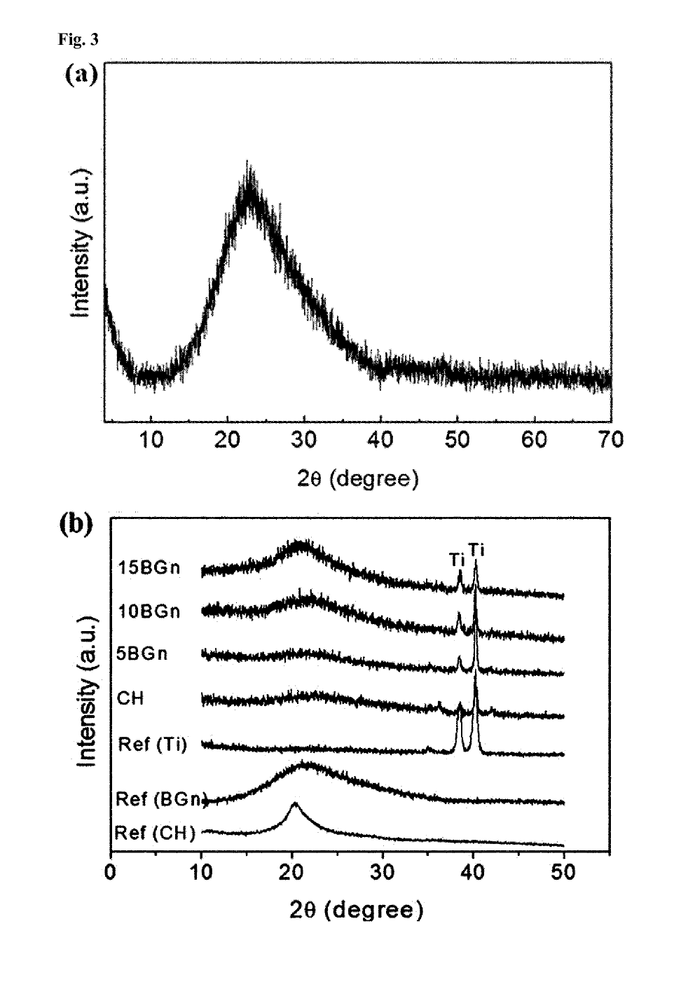

The crystalline phases of the surface aminated bioactive glass nanoparticles of Example 1) and the chitosan-bioactive glass compound coating layer of Example 2) (CH-5BGn, CH-10BGn and CH-15BGn) were analyzed by XRD (Ultima IV, Rigaku). Analysis was carried out with the voltage of 40 kV and the current of 40 mA at the diffraction angle from 10° to 50° by interval of 1°, and the results are shown in FIG. 3.

As shown in FIG. 3a, typical amorphous silica phase of the broad peak at 2θ=22.5° was identified.

In addition, as shown in FIG. 3b, the compound coating layer formed on the surface of titanium substrate (CH-5BGn, CH-10BGn and CH-15BGn) exhibited the peak only corresponding to chitosan (CH) and BGn (surface aminated bioactive glass nanoparticles), and the increased intensity of the glass indicates the combination with the inside of the coating layer.

3) FT-IR (Fourier Transform Infrared) Analysis

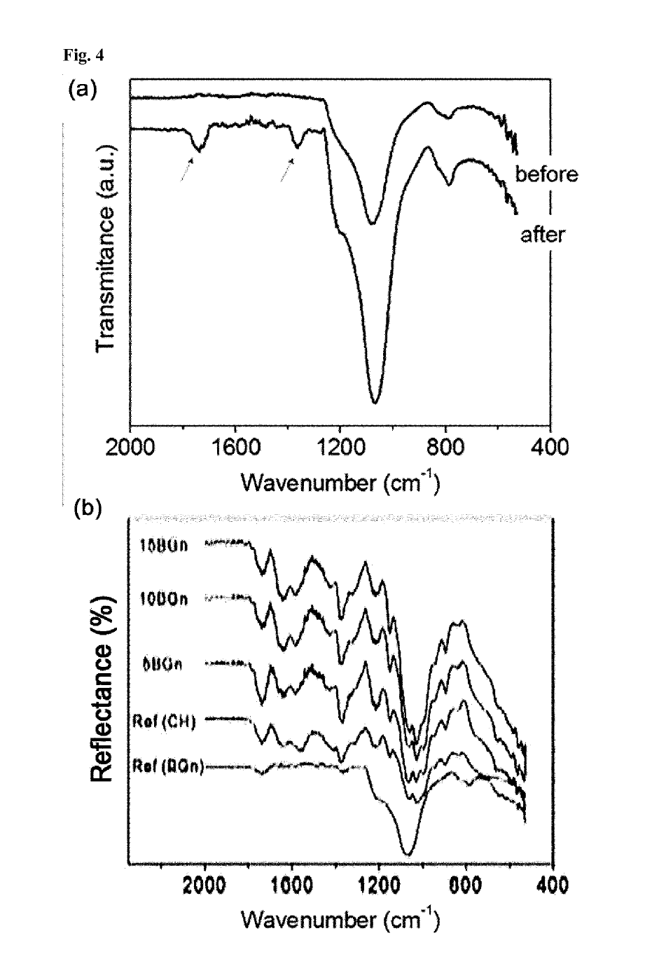

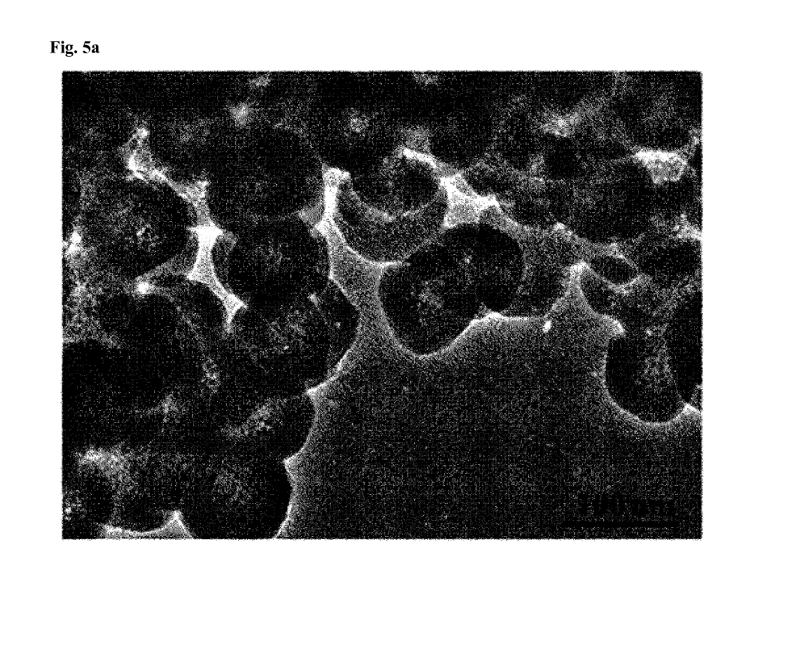

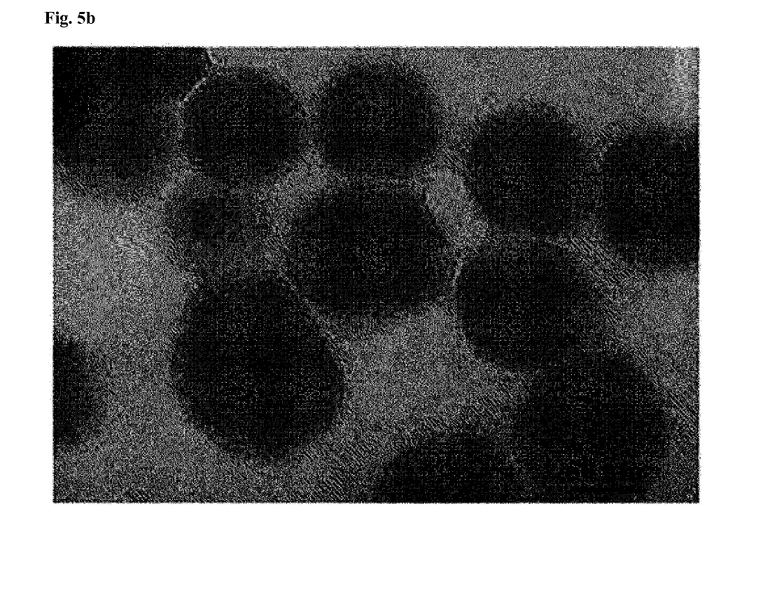

In order to identify the structure of the chemical bond of the surface aminated bioactive glass nanoparticles of Example 1) and the chitosan-bioactive glass compound coating layer of Example 2) (CH-5BGn, CH-10BGn and CH-15BGn), FT-IR (Varian 640-IR) analysis was performed. Analysis was carried out from 2000 cm⁻¹ to 500 cm⁻¹ with resolution of 4 cm⁻¹ and the results are shown in FIG. 4.

As shown in FIG. 4a, while the spectrum of bioactive glass nanoparticles which was not surface-aminated represented the bands associated with silica glass only such as 544 cm⁻¹ and 1200 cm⁻¹ (Si—O—Si bond), 1070 cm⁻¹ (Si—O—Si stretching) and 784 cm⁻¹ (Si—O—Ca vibration), the surface aminated bioactive glass nanoparticles showed the additional bands at 1365 cm⁻¹ 1737 cm⁻¹, which represent the aromatic amine-NH₂ stretching mode.

In addition, as shown in FIG. 4b, the bands (544 cm⁻¹, 1070 cm⁻¹, 1200 cm⁻¹, 1365 cm⁻¹ and 1373 cm⁻¹) corresponding to the bioactive glass nanoparticles were found to increase with increase in the bioactive glass nanoparticle content inside the compound coating layer. This result represents that coating composition (content of bioactive glass particles) and the thickness of coating layer can be easily controlled through electrophoretic deposition.

4) TEM (Transmission Electron Microscopy) Analysis

TEM analysis of the surface aminated bioactive glass nanoparticles of Example 1) and the chitosan-10 wt % bioactive glass compound coating layer of Example 2) was performed. The results are shown in FIG. 5.

As shown in FIG. 5a, the surface aminated bioactive glass nanoparticles of Example 1) formed the particles of the uniform size less smaller 100 nm (85±15 nm).

In addition, as shown in FIG. 5b, each particle was found to be separated independently by being completely surrounded by the chitosan matrix.

5) Turbidity Test

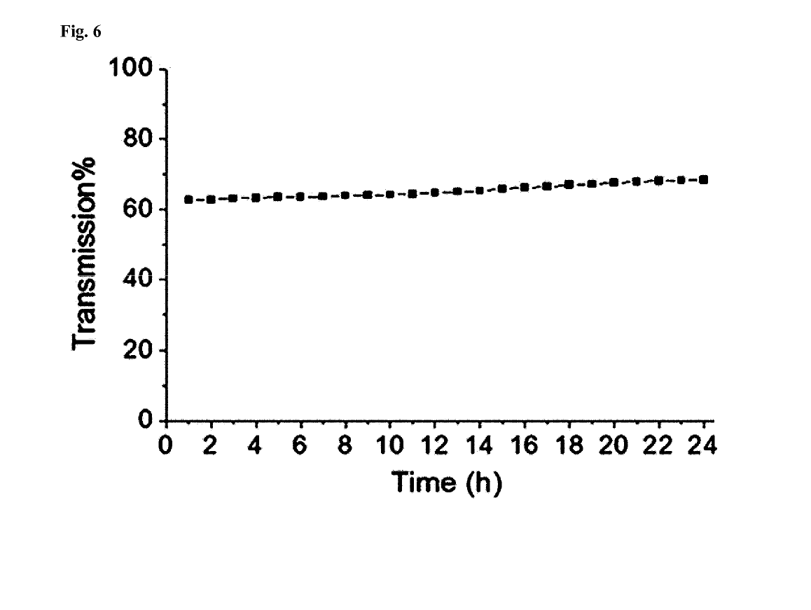

In order to identify the granular stability of chitosan-10 wt % bioactive glass complex coating composition of Example 2), turbidity test was performed. Turbidity test was performed by observing optical transmittance (%) for up to 24 hours by every hour. The results are shown in FIG. 6.

As shown in FIG. 6, optical transmittance appeared almost constant during observation time, only with a little change. This result represents that the nanoparticle complex coating composition has the high stability of the granules.

6) TGA (Thermo Gravimetric Analysis)

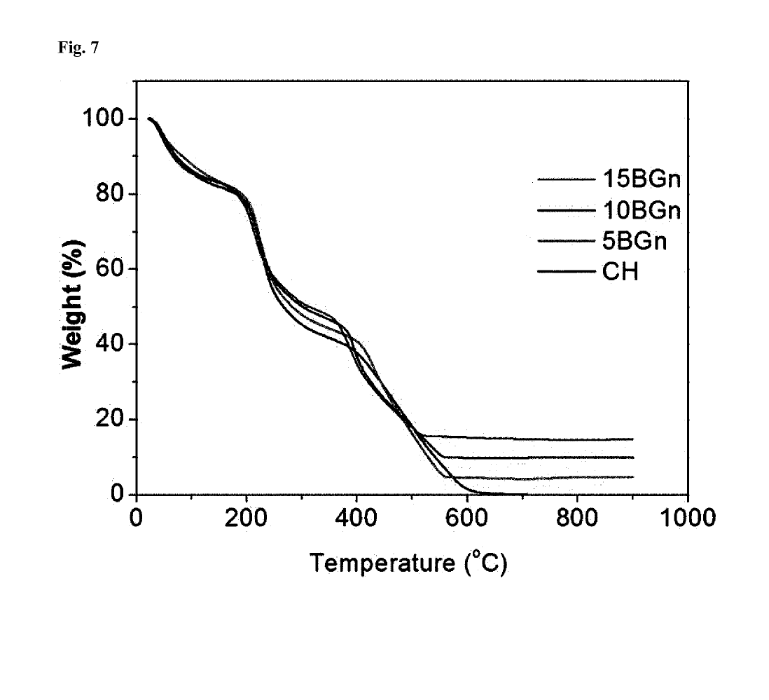

Thermogravimetric analysis (TGA, TGA N-1500, Scinco, South Korea) of the chitosan (CH, control) coating layer and chitosan-bioactive glass compound coating layer (CH-5BGn, CH-10BGn and CH-15BGn) of Example 2) was performed. Thermogravimetric analysis of deposit was measured using the part of coating layer scrapped from Titanium substrate. Thermogravimetric analysis process was adjusted to 900° C. at the heating rate of 10° C./min. Based on the same, the amount of the bioactive glass nanoparticles inside the compound coating layer was estimated. The results of thermogravimetric analysis are shown in FIG. 7.

As shown in FIG. 7, chitosan (CH) showed the mass loss in three steps. Step 1 is the loss of 22% corresponding to the release of water adsorbed to 200° C., and the subsequent steps 2 and 3 of 200° C.-350° C. and 350° C.-600° C., respectively, showed the loss corresponding to the pyrolysis of chitosan. While chitosan showed the mass loss of almost 100% at 600° C., the compound coating layer (CH-5BGn, CH-10BGn and CH-15BGn) of Example 2) showed almost similar behavior as thermogravimetric analysis pattern of chitosan but the effect of mass conservation. The measured mass conservation (residual mass) of CH-5BG, CH-10BGn and CH-15BGn is 4.89%, 9.99% and 14.84%, respectively. This result represents that the compound coating layer conserved the significant amount of initial composition.

7) SEM (Scanning Electron Microscopy) Analysis

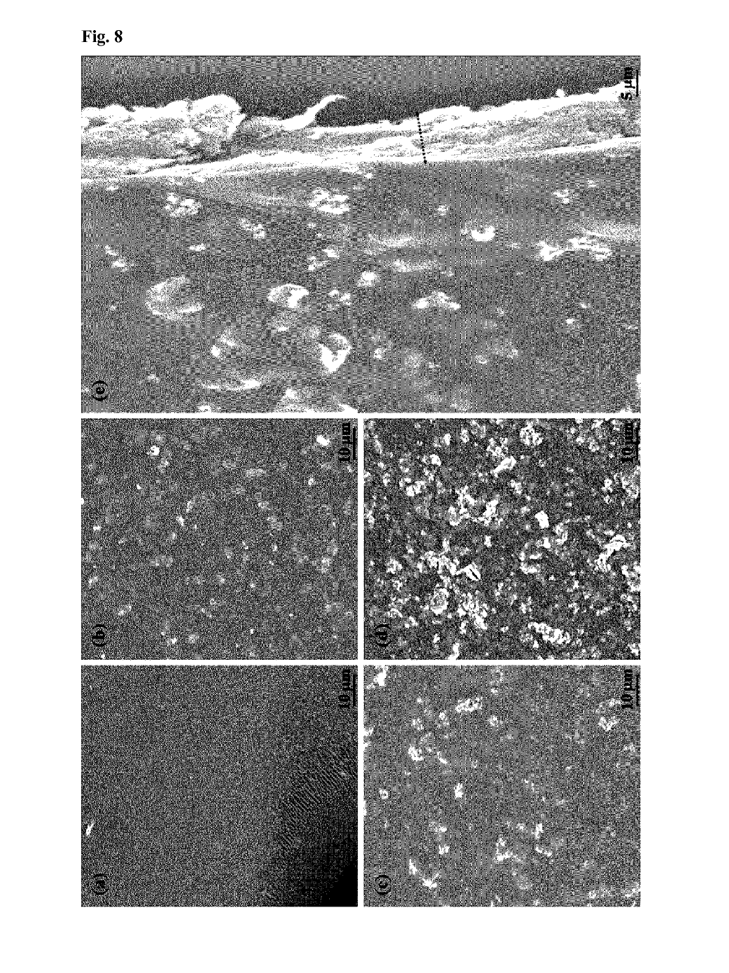

In order to identify the fine structure of chitosan (CH) coating layer and chitosan-bioactive glass compound coating layer (CH-5BGn, CH-10BGn and CH-15BGn) of Example 2), SEM (S-3000H microscope, Hitachi, Japan) analysis was performed. In addition, the approximation of the coating thickness was identified using the cross-sectional SEM images obtained from 3-5 samples of each composition. The results are shown in FIG. 8.

As shown in FIG. 8, while pure chitosan (CH) shows the coating layer of homogeneous and clear morphology, chitosan-bioactive glass compound coating layer (CH-5BGn, CH-10BGn and CH-15BGn) showed a rough surface, which was more obvious when the content of surface aminated bioactive glass particle was larger. The surface aminated bioactive glass nanoparticles were shown as the bright area of the mass of local size in micrometer (larger than independent surface aminated bioactive glass nanoparticle). Since the surface aminated bioactive glass nanoparticles in chitosan solution is relatively stable, the formation of the quasi-mass can be considered to be due to electrophoretic deposition.

Meanwhile, the morphology of the cross section (FIG. 8c) was identified by scratching off from the titanium substrate. CH, CH-5BGn, CH-10BGn and CH-15BGn showed a thickness of ˜12 μm, ˜15 μm, ˜30 μm, and ˜48 μm, respectively. This was consistent with analysis results of the weight gain of coating layer in FIG. 1c.

### Experimental Example 2

Analysis of Decomposition and Apatite Formation

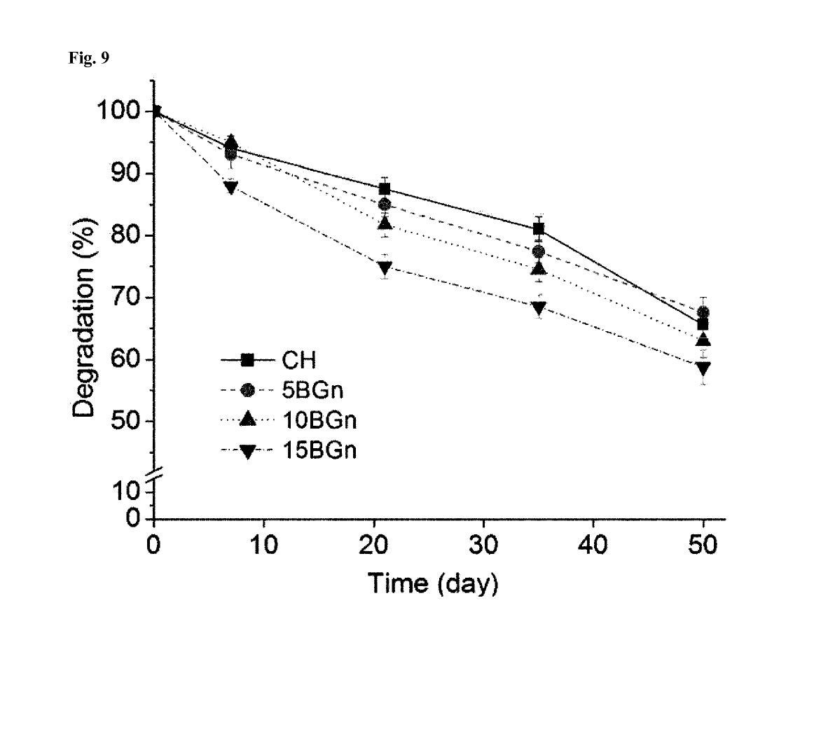

In order to determine the degree of decomposition of each coating layer (CH, CH-5BGn, CH-10BGn and CH-15BGn) prepared in Example 2), the weight change was measured after each coating layer sample (10 mm×10 mm×2 m) was immersed in the phosphate buffered saline (PBS, pH 7.4) 30 mL 37° C. for various periods (7, 21, 35, and 50 days) was taken out. The measurement results are shown in FIG. 9.

As shown in FIG. 9, all samples (CH, CH-5BGn, CH-10BGn and CH-15BGn) showed almost linear decomposition profile depending on the time, and the decomposition rate was increased by the addition of the surface aminated bioactive glass nanoparticles (BGn). In pure chitosan (CH) coating, the decomposition was found to be ˜5% after 7 days, ˜13% after 21 days, ˜18% after 35 days and ˜34% after 50 days. In chitosan-bioactive glass compound coating (CH-15BGn), the decomposition was ˜12% after 7 days, ˜25% after 21 days, ˜32% after 35 days and ˜42% after 50 days. This was due to the linear release pattern representing the decomposition of coating associated with the surface erosion process observed inside the coating layer and therefore can have a significant impact on the release of drug mixed inside of the coating layer.

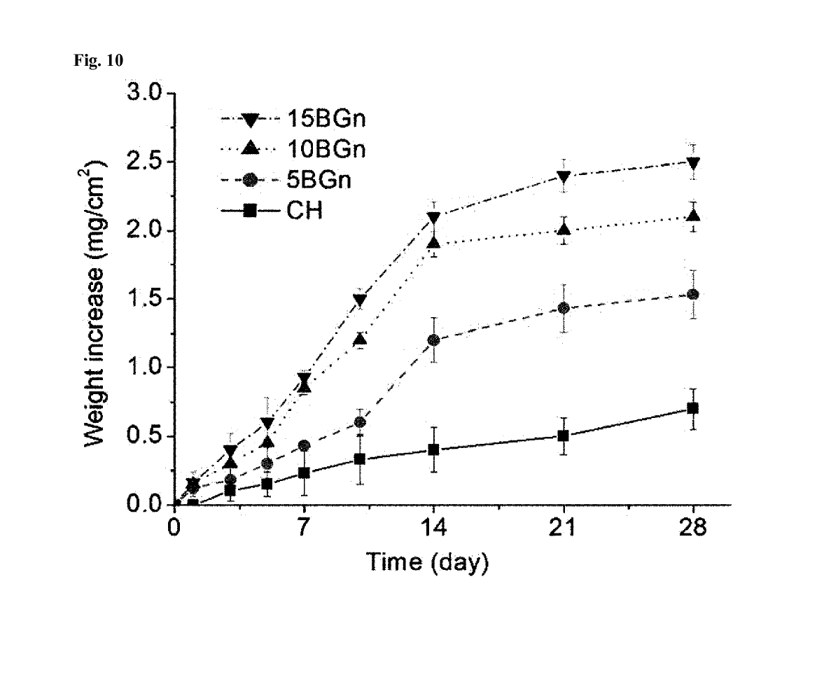

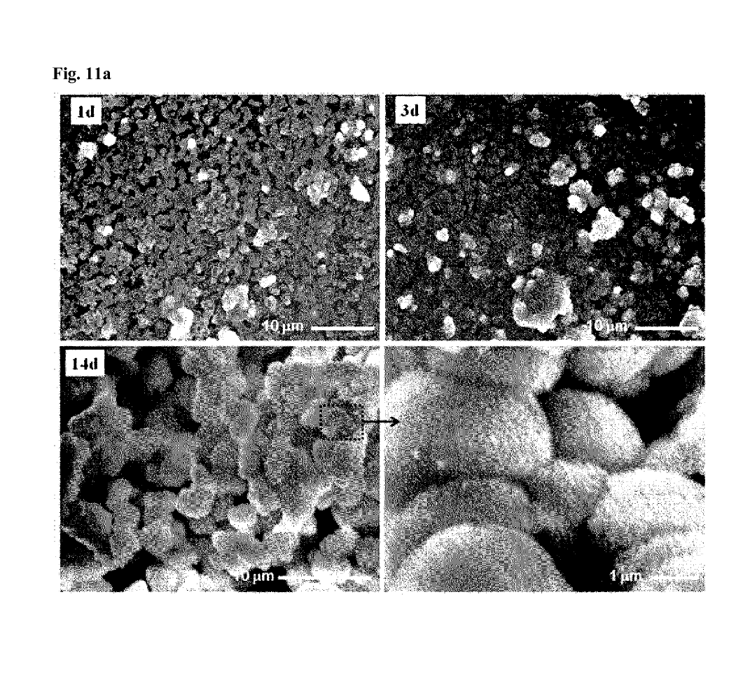

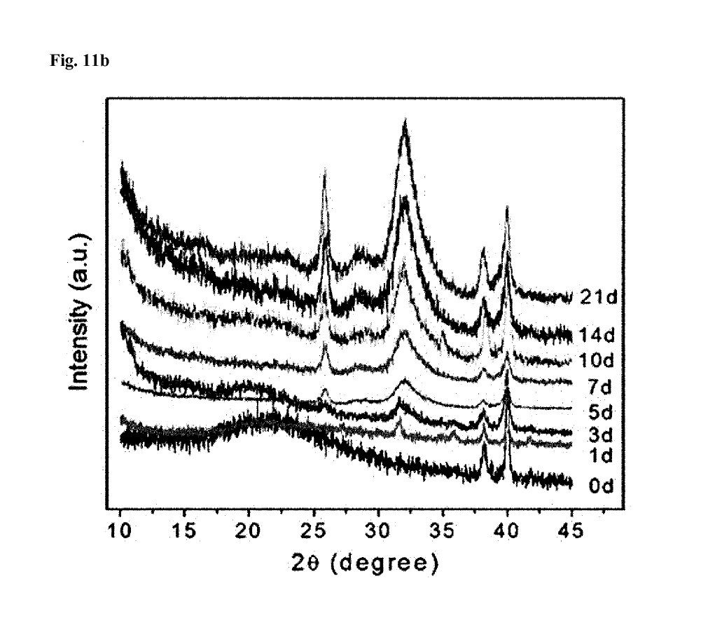

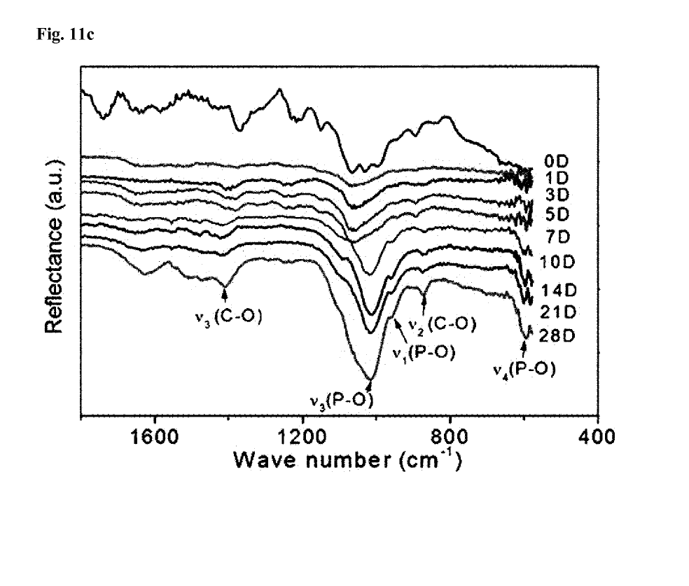

In addition, in order to identify the apatite formation capability, the 2×SBF (acceleration medium), with the ion concentration higher than simulated body fluid (SBF) by two times, was used. In this case, Na⁺, K⁺, Mg²⁺, Ca²⁺, Cl⁻, HCO₃⁻, HPO₄²⁻and SO₄²⁻ was 284.0 mM, 10.0 mM, 3.0 mM, 5.0 mM, 295.6 mM, 8.4 mM, 2.0 mM and 1.0 mM, respectively. Each coating layer sample (10 mm×10 mm×2 m) was immersed in 2×SBF of 10 mL, and then cultured at 37° C. for different periods (1, 3, 5, 7, 10, 14, 21, and 28 days). At each time, the sample was taken, washed with deionized water and then dried. Weight changes of the sample depending on apatite formation were measured. In addition, the changes in surface morphology and chemical bond structure of the sample changes were analyzed by SEM, XRD and FT-IR, respectively. Analysis results are shown in FIGS. 10 and 11.

As shown in FIG. 10, while pure chitosan (CH) showed the weight gain on day 3, chitosan-bioactive glass compound coating layer (CH-5BGn, CH-10BGn and CH-15BGn) using complex coating composition showed the weight gain from day 1. The higher content of the surface aminated bioactive glass nanoparticles showed the more noticeable difference, and the weight gain is thought to be due to the deposition of apatite mineral on the coating layer.

In addition, as shown in FIG. 11a, the surface morphology of the sample was observed while immersed in the simulated body fluid. The morphology of chitosan-bioactive glass compound coating layer (CH-10BGn) was shown as a representative sample. On day 1, several pieces of minerals were observable, and on day 3 mineral pieces were observed on the almost all surfaces. And on day 14, the mineralization to the significantly larger crystal size was observed. High magnification of the mineral phases showed the polyhedral surface of nanoparticles observed in the biomimetically mineralized apatite.

As shown in FIG. 11b, the main apatite peak at 20=32° appeared sharper and stronger with an increase in immersion time.

In addition, as shown in FIG. 11c, FT-IR spectra also showed the apatite associated peaks (596 cm⁻¹; ν₂ P—O bending, 957 cm⁻¹; ν₁ P—O, and 1018 cm⁻¹; ν₃ P—O stretching) after immersion and the intensity of band was increased according to the immersion time. Furthermore, CO₃²⁻, and ν₂ C—O and ν₃ C—O stretching vibration modes of CO₃²⁻, representing the bond of carbonate group in the apatite crystal lattice, were identified at bands 874 cm⁻¹ and 1400 cm⁻¹.

As the results of analysis, the surface aminated bioactive glass nanoparticles (BGn) were identified to play an important role in improving the apatite formation in simulated body fluid, which occurs by deposition of calcium and phosphate ions, due to the ion release property of the same to accelerate the supersaturation of solution. In addition, the pure chitosan (CH) coating showed the apatite formation according to the time, although the rate of apatite formation was lower compared to the chitosan-bioactive glass compound coating (CH-BGn). This, in a high positive potential of chitosan amine groups in the mineral medium leads to the formation of calcium ions, phosphate ions, accompanied pulls can be seen as a result. Thus, the accelerated mineralization within the inside of the compound coating (CH-BGn) can be considered as the result of the concentration or supersaturation of calcium ions released from the surface aminated bioactive glass nanoparticles (BGn) within the medium and ionic precipitation.

### Experimental Example 3

Analysis of Cell Proliferation and Osteogenesis Differentiation

In order to determine the effect of chitosan coating layer (CH, control), chitosan-bioactive glass compound coating layer (CH-10BGn) of Example 2) and titanium substrate (Ti, comparison group) on ex vivo cell growth and osteogenesis differentiation, the cell test was performed.

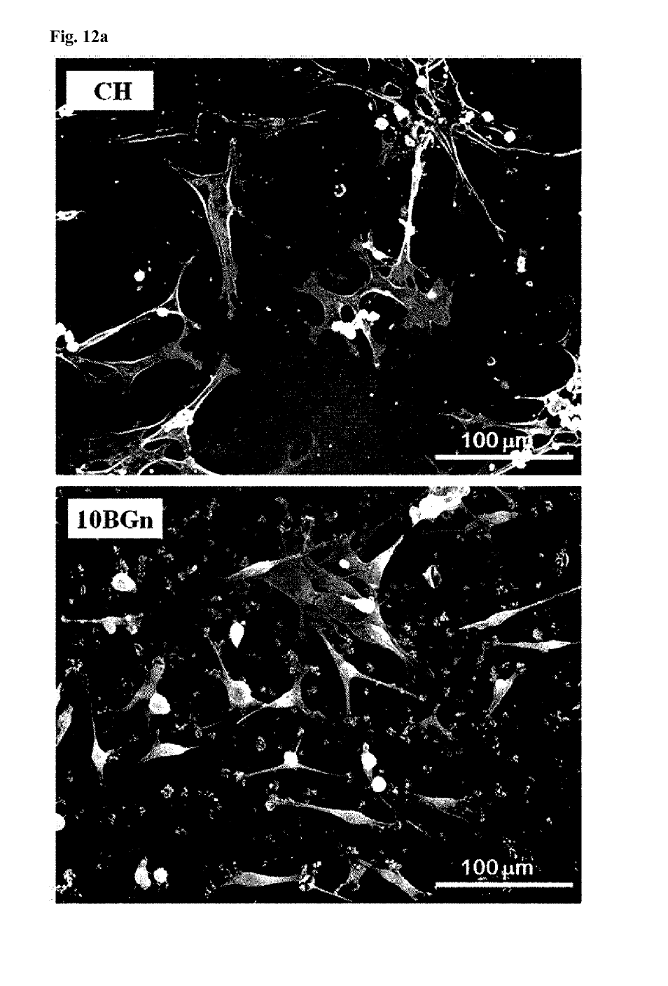

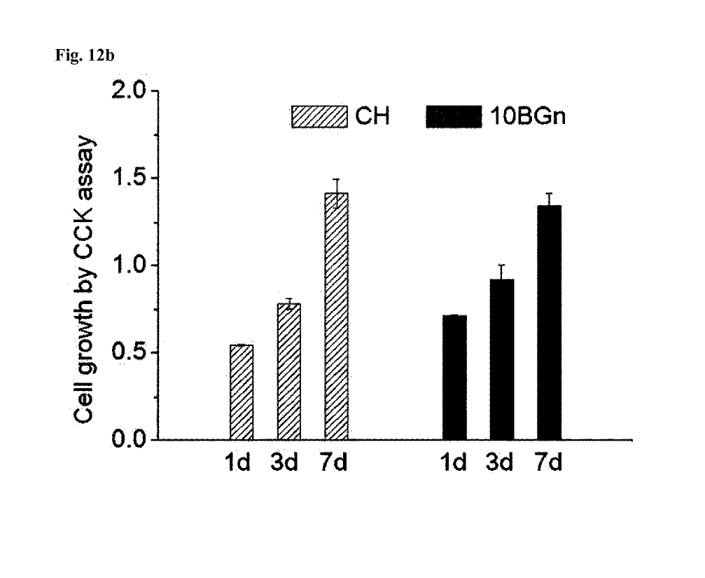

First, to analyze cell growth, each sample (CH, CH-10BGn and Ti) was sterilized with 70% ethanol and placed in the each well of 24-well plate. Pre-osteoblastic cell (MC3T3-E1; American Type Culture Collection (ATCC), USA) was smeared on the each sample with 2×10⁴ cells, and cultured in α-minimum essential medium (α-MEM; Gibco, USA) supplemented with 10% fetal bovine serum (FBS; Gibco) consisting of 1% penicillin-streptomycin at 37° C., under 5% CO₂/95% air. After incubation for 1, 3 and 7 days, the cell proliferation level was assessed through cell counting kit (CCK-8, Dojindo, Japan). In addition, after the cells were fixed in 2.5% glutaraldehyde, dehydrated with ethanol of elevated concentrations (50, 70, 90 and 100%), dehydrated in ethanol and then coated with gold, the cell morphology on the sample was observed. Experimental results are shown in FIG. 12.

As shown in FIG. 12, Mc3T3-E1 cells cultured in CH or CH-10BGn were well attached to the CH and CH-10BGn due the cytosolic process activity, and were easily dispersed. In addition, the cell growth in CH and CH-10BGn showed a continuous increase during the culture period, and based on the same, CH and CH-10BGn were identified to have useful cell growth activity as excellent cell viability.

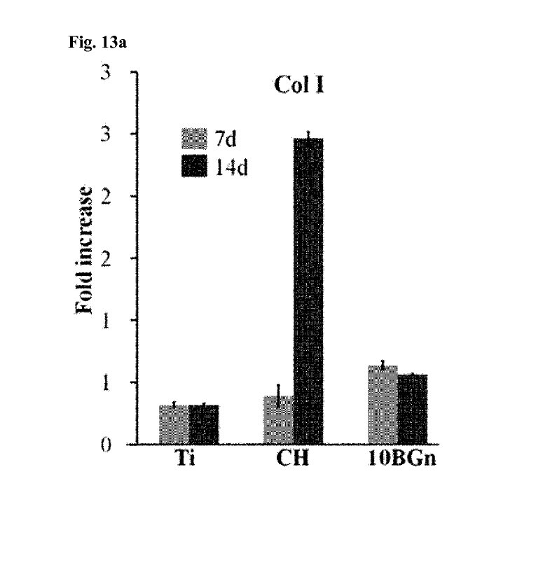

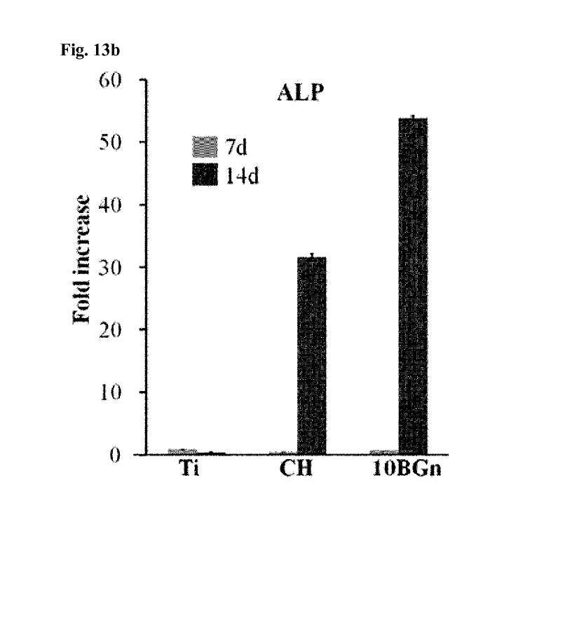

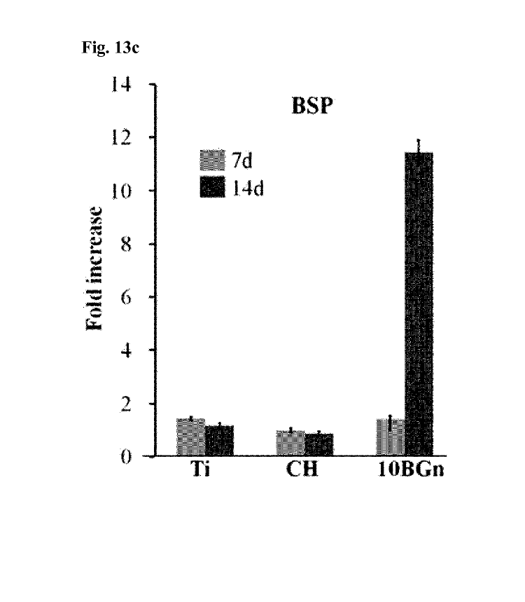

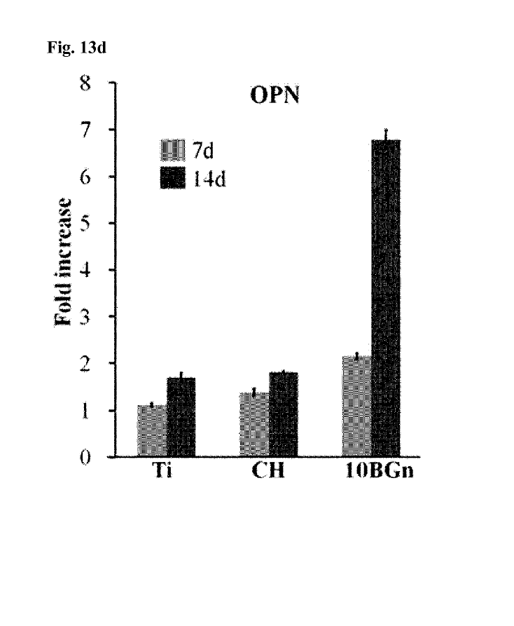

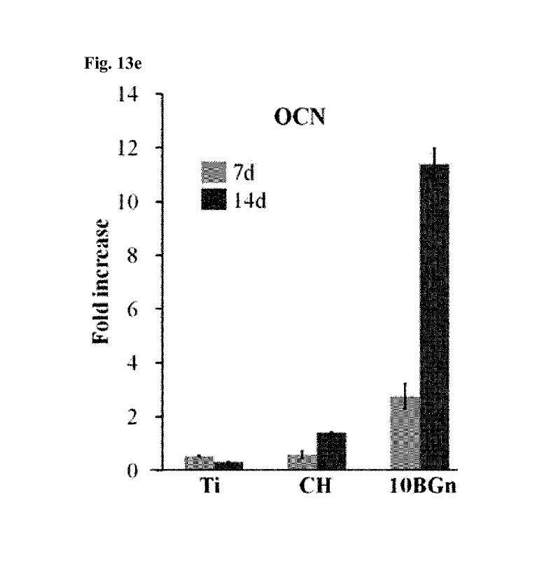

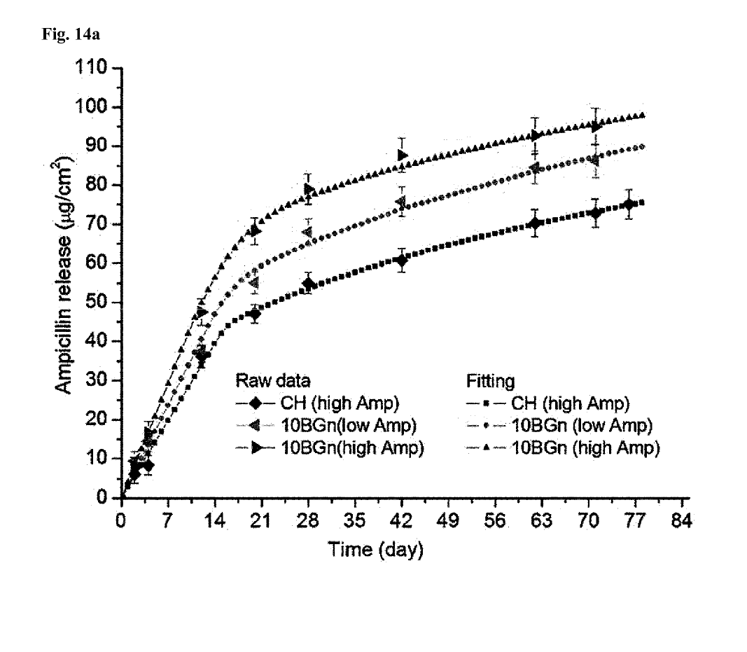

Also, in order to identify osteogenesis differentiation, the expression of bone-related genes consisting of collagen type I (Col I), alkaline phosphatase (ALP), BSP (bone sialoprotein), OPN (osteopontin) and OCN (osteocalcin) was evaluated. After 7 days and 14 days of incubation, total RNA was extracted from cells using RNeasy Mini kit (Qiagen, South Korea). Total RNA of 2 μg was used to perform the reverse transcriptase (RT) reaction. Real-time polymerase chain reaction (PCR) was performed in Rotor-Gene 3000 spectrofluorometric thermal cycler (Corbett Research, Australia) using SYBR Green PCR kit (Quantace, GCbiotech, Netherlands). After PCR performed, Ct values were used to measure the ability of other genes compared to β-actin which is used as an internal control substance (ΔCt=Ct gene−Ct β-actin). Then mRNA within the each sample was calculated as the relative ΔΔCt (ΔCt gene−ΔCt β-actin) values. Sense and antisense primer was designed according to the published cDNA sequence available from GenBank. Each measurement was performed three times. Analysis results are shown in FIG. 13.

As shown in FIG. 13, whereas the gene expression was relatively low on day 7, the expression level was identified to be adjusted as high in CH-10BGn on day 14. In all the other genes (ALP, BSP, OPN, and OCN) except for collagen type I (Col I), CH-10BGn showed the significantly higher gene expression than comparison groups (Ti) and the control group (CH).

The experimental results identified that the addition of surface aminated bioactive glass nanoparticles (BGn) has an effect mainly on stimulating osteogenesis differentiation rather than accelerating cell growth (proliferation). During the incubation period of several weeks, the coating layer was decomposed over time (FIG. 9). Ionic products such as calcium and silicon separated from BGn of CH-BGn, in addition to CH, by melting have contributed to improve osteogenesis. Addition of BGn or ions separated from BGn significantly promoted osteogenesis differentiation including the gene expression in one osteoblast or mesenchymal stem cells, protein synthesis and mineral formation.

### Experimental Example 4

Analysis of Drug Delivery and the Antibacterial Effect of Drug Delivery Layer

Na-ampicillin emission test of the drug delivery layer sample (CH (high Amp), CH-10BGn (low Amp) and CH-10BGn (high Amp)) prepared in Example 3) was performed. A release test was performed in PBS of pH 7.4, 37° C. Each sample was cultured in PBS during different period up to 10-11 weeks. At each time, the sample was taken and absorbance changes at the characteristic wavelength of 230 nm wavelength were analyzed by monitoring the solution consisting of the released Na-ampicillin by UV-VIS spectroscopy using Libra S22 apparatus (Biochrom, UK). A series of deionized water (10-100 μg/mL) consisting of standard Na-ampicillin solution were prepared and the linear calibration curve (R2=0.99) was obtained using the Beer's law Equation 1.

A=abc [Equation 1]

Wherein, A is the absorbance, a is a constant known as the extinction coefficient, c is the concentration, and b is the cell bath length (constant).

In order to remove any possible interference of decomposition product, co-solution for UV—spectral analysis was prepared by collecting the solution obtained from the coating without Na-ampicillin during the culture time same as drug-eluting period.

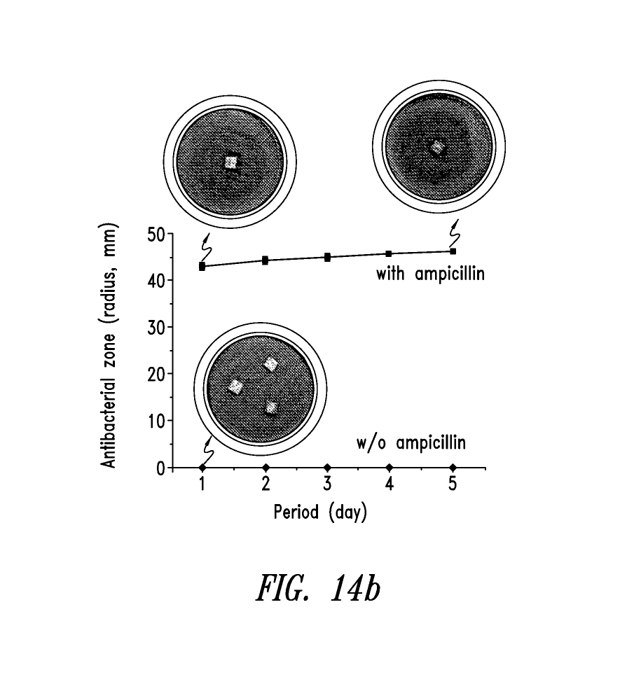

In addition, the antibacterial effects of Na-ampicillin released from CH-10BGn were investigated by performing the agar diffusion tests for streptococcus mutants (ATCC, USA). The coating samples with or without Na-ampicillin (CH-10BGn (with Amp) and CH-10BGn (free Amp)) were used. Streptococcus mutants 100 mL were smeared directly on the agar plate, cultured overnight at 37° C., each sample was placed on the agar plate, and then inhibition zone formed by Na-ampicillin released from the coating layer was observed at 24 hours interval for 5 days. The results of drug release test and the antibacterial effect analysis are shown in FIG. 14.

As shown in FIG. 14a, the drug release pattern from each drug delivery layer sample (CH (high Amp), CH-10BGn (low Amp) and CH-10BGn (high Amp)) was gradual at the initial stage, and showed a consistently high durability until 11 weeks. Despite the maximum value at 11 weeks during the experimental period, the sustained release pattern at 11 indicates the possibility of continuous release beyond the experimental period. Accordingly, the drug delivery layer of the present invention can be usefully applied as drug delivery layer for the long-term release at the almost constant release rate.

Meanwhile, the drug delivery layer (CH-10BGn (low Amp) and CH-10BGn (high Amp)) of the present invention showed higher drug release effect compared to the CH drug delivery layer control group. The release pattern in FIG. 14 showed the patterns in two steps, which are the linear step until initial 14 days and the subsequent parabola-like pattern. Therefore, for more accurate identification, the parameters according to the pattern (release rate constant and release index) were calculated. Equation 2 which is the zero-order model was used for the linear first step and Equation 3 which is Riteger-Peppas empirical equation was used for the second parabola-like pattern.

$$\begin{matrix}\frac{{M}_{t}}{{M}_{∞}} = {K}_{0} t & [ Equation 2 ] \\ \frac{{M}_{t}}{{M}_{∞}} = {Kt}^{n} & [ Equation 3 ]\end{matrix}$$

In Equation 2 and Equation 3, Mₜ and M_{∞} are the absolute amount of drug released at time t and infinite time (∞), respectively, K₀ and K are the release rate constants that reflect the structural and geometric characteristics of the drug delivery device in each Equation, and n is the release index which indicates the drug release mechanism. The parameters calculated from the release pattern curve in FIG. 14a are shown in Table 1.

TABLE 1

| | CH | | |
|---|---|---|---|
| Section | (high amp) | CH-10BGn (low Amp) | CH-10BGn (high Amp) |
| K₀ | 2.82 | 3.38 | 4.16 |
| K | 17.5 | 22.6 | 35.2 |
| n | 0.44 | 0.37 | 0.38 |

As shown in Table 1, the initial step represented a linear pattern with R2 value lower than 0.99 and the second step also represented the strong release indices, which are 0.44, 0.37, and 0.38 for CH (high Amp), CH-10BGn (low Amp) and CH-10BGn (high Amp), respectively.

In addition, as shown in FIG. 14b, Na-ampicillin-containing CH-10BGn showed antibacterial effect at the time when the experiment day 1 passed (24 hours), which was maintained for 5 days. However, in CH-10BGn without the drug, the antibacterial effect could not be identified.

What is claimed is:

1. A method for preparation of an implant comprising a drug delivery layer comprising
1) preparing a chitosan-bioactive glass composite solution by dispersing chitosan solution in solvent and adding surface aminated bioactive glass nanoparticles;
2) preparing a drug-containing complex coating composition by adding a drug to the chitosan-bioactive glass composite solution; and
3) preparing a drug delivery layer by electrophoretic deposition of the complex coating composition on the surface of the implant.

2. The method according to claim 1, wherein the surface aminated bioactive glass nanoparticle in step 1 is prepared through the process comprising
preparing a calcium nitrate-template mixture solution by preparing PEG template solution and adding calcium nitrate;
preparing a reaction product by adding TEOS solution in calcium nitrate-template mixture solution, sonicating and stirring;
preparing the bioactive glass nanoparticles by centrifuging the reaction product, washing and calcining; and
adding APTES followed by refluxing after adding the prepared bioactive glass particles in a solvent followed by dispersing.

3. The method according to claim 1, wherein the surface aminated bioactive glass nanoparticles comprise silica (SiO₂) and calcium oxide (CaO) in a molar ratio of 85:15.

4. The method according to claim 1, wherein the chitosan solution in step 1) is prepared by dissolving chitosan in acetic acid or hydrochloric acid solution.

5. The method according to claim 1, wherein the solvent in step 1) is the co-solvent which is the mixture of ethanol and water in a volume ratio of 1:1 to 1:9.

6. The method according to claim 1, wherein the drug-containing complex coating composition in step 2) has pH 3.1 to pH 3.6.

7. The method according to claim 6, wherein the pH is adjusted by acetic acid or sodium hydroxide.

8. The method according to claim 1, wherein the electrophoretic deposition in step 3) is performed with the DC voltage of 20 V to 80 V for 5 minutes to 10 minutes.

9. The method according to claim 1, wherein, the drug is an antibiotic, anti-inflammatory drug, anticancer drug, or bone differentiation drug.

10. The method according to claim 1, wherein the thickness of the drug delivery layer is 2 μm to 50 μm.

11. An implant composition for living donor transplantation comprising the implant prepared by the preparation method of claim 1.
